(19) United States
(12) Patent Application Publication
(10) Pub. No.: US 2021/0263025 A1
(43) Pub. Date: Aug. 26, 2021

(54) MICRO-SEPARATION FOR MULTIPLEXING

(71) Applicant: FemtoDx, Beverly Hills, CA (US)

(72) Inventors: Jiangang John Du, Granada Hills, CA (US); Robert DiNello, Alhambra, CA (US); Pritiraj Mohanty, Los Angeles, CA (US)

(73) Assignee: FemtoDx, Beverley Hills, CA (US)

(21) Appl. No.: 17/180,203

(22) Filed: Feb. 19, 2021

Related U.S. Application Data
(60) Provisional application No. 62/979,342, filed on Feb. 20, 2020.

(51) Int. Cl. G01N 33/543; B01L 3/00

(52) U.S. Cl. CPC G01N 33/54373; B01L 3/502; B01L 2400/0406; B01L 2200/026; G01N 2333/4737

(57) ABSTRACT

Articles and systems for separating micro-sized analytes.

### RELATED APPLICATIONS

This application claims priority to U.S. Provisional Application No. 62/979,342, filed Feb. 20, 2020, which is incorporated herein by reference in its entirety.

### TECHNICAL FIELD

Articles and systems for separating micro-sized analytes are generally described.

### SUMMARY

The techniques described herein provide various articles and systems for separating micro-sized analytes from a fluid sample.

In some embodiments, antigen binding entities (e.g., antibodies) are attached within a capillary tube. These antigen binding entities may bind a specific antigen and separate the antigen from a sample flowing through the capillary tube. In some examples, the antigen binding entities remain fixed within the tube, while in other examples the antigen binding entities are configured to release from the capillary tube.

In some embodiments, a plurality of tubes is connected through a junction. For example, an inlet or outlet tube can be connected to a junction, whereby the junction is in-turn connected to a plurality of outlet/inlet tubes (e.g., a first inlet tube, a second inlet tube, a first outlet tube, and/or a second outlet tube). Each outlet/inlet tube may separate a species from a sample flowing through the system and may reach a bio-specimen receiving site, for example, for further analysis by a bioassay.

The subject matter of the present invention involves, in some cases, interrelated products, alternative solutions to a particular problem, and/or a plurality of different uses of one or more systems and/or articles.

In one aspect, a capillary tube comprising a first section located within the capillary tube, the first section comprising a first material, a plurality of species-binding entities attached to the first material, and a sensor adjacent to at least some of the plurality of species-binding entities is described.

In another aspect, a capillary tube comprising a first section located within the capillary tube, the first section comprising a first material, a first plurality of species-binding entities attached to the first material wherein the plurality of species-binding entities are configured to selectively release from the first material, and a sensor proximate a distal end of the capillary tube is described.

In another aspect a capillary tube, comprising a first section located within the capillary tube, the first section comprising a first material and a first plurality of species-binding entities attached to the first material wherein the first plurality of species-binding entities is configured to selectively release from the first material, a second section located within the capillary tube, the second section comprising a second material, wherein the second material is different than the first material and configured to prevent attachment of the first plurality of species-binding entities, a third section comprising a second plurality of species-binding entities configured to selectively bind a species, the species bound to the first plurality of species-binding entities, and a sensor adjacent to at least some of the second plurality of species-binding entities is described. In yet another aspect, a system, comprising a capillary tube comprising a proximal end and a distal end wherein the capillary tube comprises a plurality of species-binding entities attached to at least a first section of the capillary tube, a first electrode proximate the proximal end, a second electrode, and a power supply connected to the first electrode and the second electrode is described.

In some embodiments, a system is described, where the system comprises an inlet tube, a junction fluidically connected to the inlet tube, and a first outlet tube, a second outlet tube, and a third outlet tube each fluidically connected to the junction wherein the inlet tube, the first outlet tube, the second outlet tube, and the third outlet tube are no greater than approximately 1 micron in a cross-section diameter.

In yet another aspect, a system, comprising an inlet tube, a junction fluidically connected to the inlet tube, and a first outlet tube, a second outlet tube, and a third outlet tube each fluidically connected to the junction wherein the inlet tube, the first outlet tube, the second outlet tube, and the third outlet tube are no greater than approximately 100 nanometers in a cross-section diameter is described.

In yet another aspect still, a system is described that comprises a first source comprising a first inlet, a second source comprising a second inlet, a third source comprising a third inlet, a junction fluidically connecting the first inlet, the second inlet, and the third inlet, an outlet tube fluidically connected to the junction, and a receiving site proximate to a terminal end of the outlet tube, wherein the first inlet, the second inlet, the third inlet, and/or the outlet tube have a cross-sectional diameter of no greater than approximately 1 micron.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C depict schematic illustrations of a plurality of species-binding entities (e.g., antibodies) binding a species (e.g., antigens) within a capillary tube, according to some embodiments;

FIG. 2 is a schematic of antibodies configured to release from a capillary tube when an antigen binds to the antibodies, according to certain embodiments;

FIG. 3 schematically illustrates a plurality of antigens bound to antibodies, where the antibodies are configured to release from a location within a capillary tube and bind to immobilized antibodies at a distal location within the capillary tube to form a sandwich-like structure, according to some embodiments;

FIG. 4 is a schematic illustration of capillary tube configured for capillary electrophoresis, where antibodies are configured to release from a section within the capillary tube and can be used to facilitate separation of analytes, according to some embodiments;

FIG. 5 is a schematic illustration of an inlet tube connected to a junction with the junction connected to a plurality of outlet tubes and bio-specimen receiving sites, according to some embodiments;

FIG. 6 is a schematic illustration of an inlet tube connected to a junction where the junction is connected to a plurality of outlet tubes and bio-specimen receiving sites that are spaced disparately, according to some embodiments;

FIG. 7 is a schematic illustration of a general system for micro/nano-aliquoting with an inlet tube connected to a junction and where the junction is connected to a plurality of outlet tubes and bio-specimen receiving sites with outlet tubes arranged proximate to control valves, according to some embodiments;

FIG. 8 is schematic showing a plurality of bio-specimen sources with valves controlling the connection to a junction where several species combine at the junction into a bio-specimen receiving site, according to some embodiments; and

FIG. 9 is a plot of the signal generated by certain concentrations of CRP protein within different zones of a capillary tube configured with antigen binding species within each zone, according to one embodiment.

### DETAILED DESCRIPTION

Articles (e.g., capillary tubes) and systems described herein may be used to separate a species (e.g., an analyte, an antigen) from a sample.

As described further herein, the techniques provide for a plurality of species-binding entities (e.g., antibodies) located within a section of a tube (e.g., a capillary tube). These species-binding entities may bind to a specific species within a sample thereby separating it from the bulk sample. Because of the specificity of the species/species-binding entity pair, if a mixture of species is present within a sample, the species-binding entity may separate a specific species from the sample while allowing the other species within the sample to continue to flow along the capillary tube without being bound by the plurality of species-binding entities. In this way, the specific species may be separated from other species within the sample that do not have specificity for the species-binding entity and, therefore, do not bind to the specific species-binding entity attached within a section of the capillary tube. In some embodiments, the plurality of species-binding entities can be configured to release from a section of the capillary tube. The release may occur when a species binds to the species-binding entity. In certain embodiments, when a plurality of species-binding entities is initially attached and then releases from within the capillary tube, a second plurality of species-binding entities may be present in a location downstream (e.g., a distal location along the direction of flow) and may bind the species/species-binding entity pair.

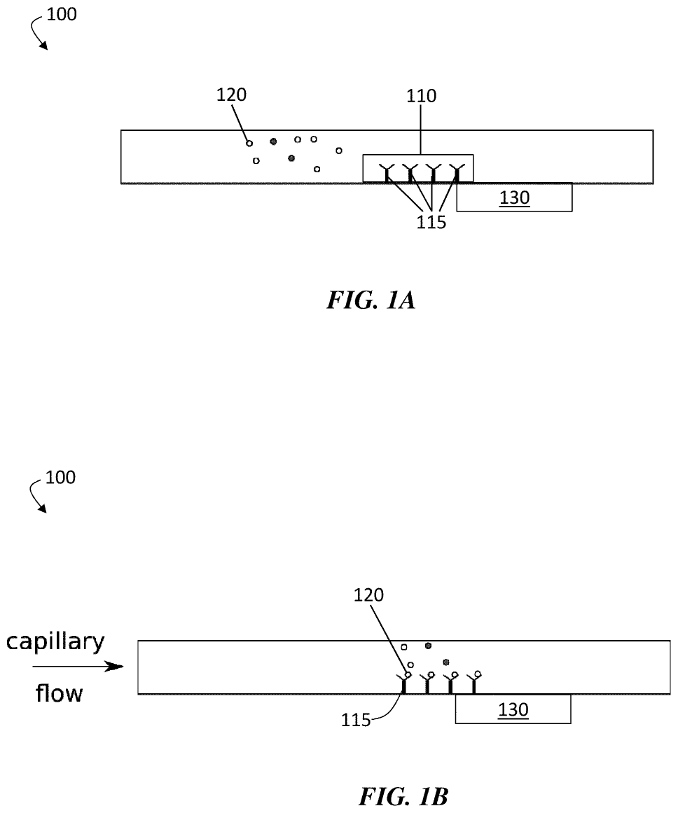

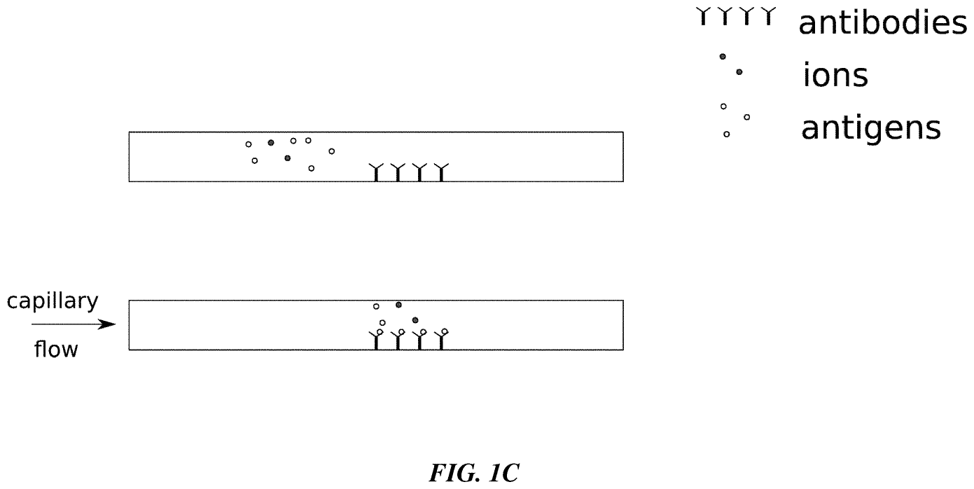

Some embodiments provide for antigen binding entities immobilized at one or more locations within a tube. The tube can be manufactured in different ways without departing from the techniques described herein. In some embodiments, a capillary tube may be a continuous tube (e.g., fabricated of the same material along the length of the tube) with antigen binding entities immobilized at one or more specific locations within the capillary tube. In some embodiments, the capillary tube may be made from a plurality of sections that may be made of the same and/or different materials. Regardless of how the tube is fabricated, antigen binding entities may be immobilized on one or more sections of the tube, and one or more other sections of the tube (e.g., the remainder of the tube) may not contain any immobilized antigen binders, as schematically illustrated in FIG. 1. Generally, in operation a sample containing an antigen can flow through the capillary tube by capillary action over the section (e.g., zone or zones) comprising specific antibody binding entities. Antigens, if present in the sample, bind to the antigen binding entities.

Some embodiments can include a sensor to detect the presence of an antigen. For example, binding of an antigen to the antibody may be detected and quantitated by changes in electric field generated by antigen binding. That is to say, when an antigen binds to an antigen binding entity, formation of an antigen-antibody complex may result in a change in an electric field, which may be sensed using a sensor. In some embodiments, a sensor detects antigen(s) binding to an antigen binding entity(ies) (e.g., an antibody). In some embodiments, changes in surface plasmon resonance reflectance intensity generated by antigen binding and other techniques known to those skilled in the art may be used to detect and quantitate antigen binding.

As described herein, an antigen binding entity can be disposed in a single section (e.g., zone) of a capillary tube. In such an embodiment of a single zone for comprising antigen binding entities, the antigen may be quantitated by construction of a standard curve of assay response versus antigen concentration, as will be described below in Example 1. In the case of multiple zones of antigen binding entity, the antigen may be quantitated by constructing a standard curve of assay response versus antigen concentration for each zone and the results averaged.

In some embodiments, when the concentration of the antigen is very high, one or more zones may be saturated with antigen and no longer able to bind to additional antigen. In such embodiments, as the sample moves up the tube by capillary action, antigen is progressively removed from the solution by each antigen binding section or zone until a section or zone is not saturated by antigen and can, again, be used to quantitate the antigen.

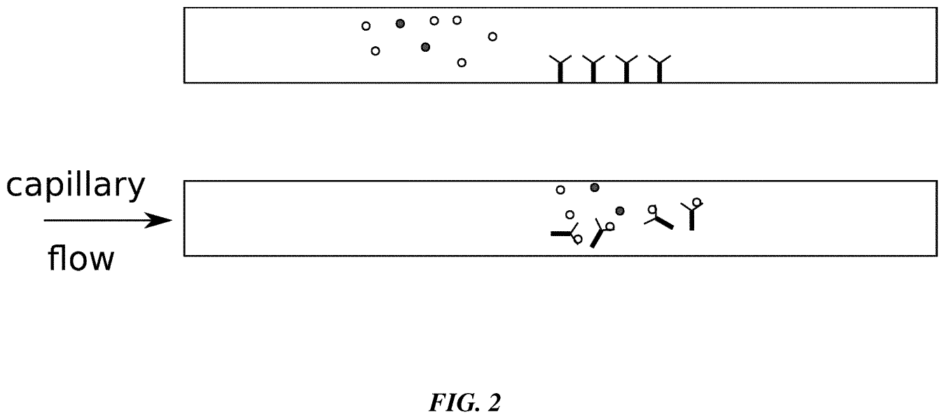

Some embodiments relate to antigen binding entities attached to one or more sections of a tube in a manner such that the antigen binding entities can be released from the tube (e.g., upon binding of antigens). As described herein, the capillary tube may be a continuous tube of the same material. The material chosen for a continuous tube of the same material can be a low protein binding material such that antigen binding entities deposited on the inside surface of the capillary are not firmly bound and, upon exposure to aqueous sample solutions, these antigen binding entities become soluble and move down the tube by capillary flow. That is to say, in certain embodiments, antigen binding entities may be attached to a section within a capillary tube, but may subsequently be released from the section depending on the material of the section, and in some embodiments, binding of an antigen to the antigen binding entities may cause the release of the antigen binding entities from the section, as schematically illustrated in FIG. 2. In some cases, the material for the capillary tube may be chosen for other desirable properties without respect for its ability to bind proteins. In such an embodiment, the tube is treated with materials, known to those skilled in the art, which can block binding of proteins to the walls of the capillary tube, thus ensuring that the antigen binding entity will be released upon exposure to sample.

As described herein, a capillary tube may contain multiple sections within the tube. The antigen binding entities may be deposited upon on one or more sections comprised of low protein binding material and other sections may be of other materials chosen for other desirable properties without respect to protein binding. If a material desirable for use in a multi-section capillary tube binds protein (e.g., an antigen), then other sections may be treated with materials known to block protein binding.

As described herein, in some embodiments a sample flows up the capillary tube to one or more sections (e.g., zone(s)) comprised of antigen binding entities of a single specificity. Antigen, if present, binds to the antigen binding entities, which are simultaneously released from the tube wall and move along the tube by capillary flow. Single antigen binding entity-antigen complexes exiting the tube from a point distal to the sample application point may be quantitated by methods known to those skilled in the art.

Some embodiments relate to multiple antigen binding entities of different specificities that are each immobilized in a different zone on the inside of the tube (e.g., a continuous capillary tube). As a sample moves along the capillary tube, each antigen binding entity can bind its specific antigen and can simultaneously be released from the capillary tube wall and move along the tube by capillary action. The mixture of antigen binding entity-antigen complexes exiting the tube from a point distal to the sample application point may be quantitated by methods known to those skilled in the art.

In some embodiments, some features described above and/or herein may be combined into an assay system for the detection of one or multiple analytes. A sample is applied to one end of the capillary tube, the proximal end, and liquid exits the capillary tube from the distal end. As described herein, the capillary tube may be a continuous tube of the same material and/or may be a capillary tube comprising one or more sections (e.g., of the same and/or different materials). The material chosen for the continuous tube of the same material can be a high protein binding material (e.g., which can attach antigen binding entities that are immobilized to the high protein binding material). If it is desired that sections of the tube have low protein binding capacity (e.g., antigen binding entities may release from the low protein binding material), then the sections can be treated with materials, known to those skilled in the art, which can block binding of proteins to the walls of the required sections of the capillary tube. In some embodiments, such as the case of a multi-section tube, the various sections of the tube can be low protein binding or high protein binding, as desired. If only sections which exhibit low protein binding can be used, however, then the required sections can be treated with materials, known to those skilled in the art, which block binding of proteins to the walls of the capillary tube.

In some embodiments, antigen binding entities of a given specificity are applied to a low protein binding zone closer to the proximal end of the tube and farther from the distal end. Unless required for optimal assay performance, multiple labeled antigen binding entities can be applied as a mixture to one low protein binding zone closer to the proximal end of the capillary, thus simplifying the process of manufacturing the functionalized capillary tube. Antigen binding entities applied closer to the proximal end of the tube may contain a detectable label. Labels used may include, but are not limited to, enzymes, nanoparticles (such as gold, selenium, silver or iron nanoparticles), chemiluminescent compounds, fluorescent compounds, europium chelates and electro-chemiluminescent labels. A signal may be measured using a sensor that comprises a spectrophotometer, a fluorimeter, a luminometer, surface plasmon resonance measurement and changes in electric field as non-limiting examples.

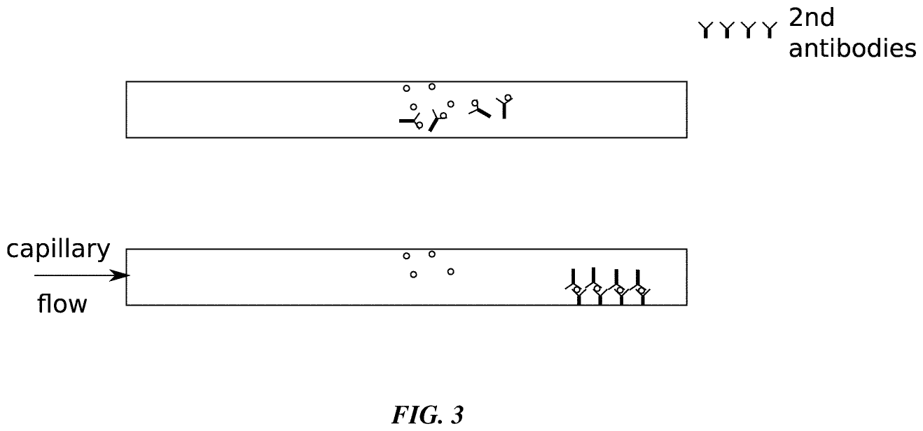

Unlabeled antigen binding entities of the same specificity, which can tightly bind to the tube in a section of high protein affinity, may be applied to sections (e.g., zones) closer to the distal end of the tube and farther away from the proximal end. Multiple analytes may be determined in a single capillary tube by binding each unlabeled antigen binding entity in a certain zone closer to the distal end of the tube. As the assay progresses, the sample reaches the zone containing the labeled antigen binding entities at the proximal end of the capillary tube. In some embodiments, the antigen binding entities are solubilized and, at the same time, bind their specific antigens, if present such that these antigen binding species are released from a section of the capillary tube. As the sample containing the complexes of antigen and labeled antigen binding entities reaches the distal end of the tube, each complex binds to a section containing the immobilized (tightly bound) antigen specific entity which recognizes the antigen bound to the labeled antigen-specific binding entity forming a sandwich-like structure, as schematically depicted in FIG. 3. A sensor can be used to determine the binding, as described herein. In this way, multiple antigen concentrations in the sample can be determined in a single test by measuring the signal from each zone specific to a certain antigen(s). Sample volume required for capillary based tests are typically small, 10 ul or less and can be easily and less painfully obtained than larger volume samples.

In some embodiments, quantitative assay results may be obtained by determining a standard curve using antigen spiked into the biological fluid to be used in the test. Results for each zone can be determined by one of the methods described herein and plotted as a signal on the X axis and antigen concentration on the Y axis, such that an appropriate curve fitting program can be used to determine the equation of the curve. This can be done for each zone of tightly bound antigen specific entity.

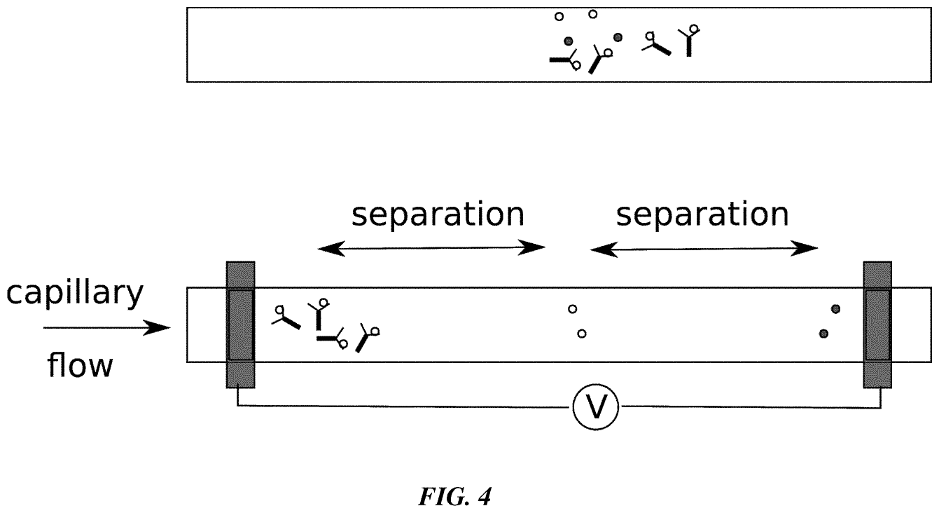

According to some embodiments, capillary tubes with species-binding entities attached to sections within the capillary tube may be a part of a capillary electrophoresis system. The system may comprise a capillary tube, an anode, a cathode, a power source connected to the anode and the cathode, and may be used to separate different analytes within the capillary tube. However, the inventors have recognized and appreciated that inclusion of species-binding entities with an affinity for certain analytes may improve the separation obtained compared to existing capillary electrophoresis systems. Fluid carrying bio-specimens (e.g., fluid-borne antigens, antibodies and antigens) may flow in a capillary tube (or a plurality of capillary tubes) by capillary action. Segments of tube/channel can be equipped with electrodes (e.g., an anode, a cathode) on both ends and a static electric field can be applied through electrodes. A schematic of such is shown in FIG. 4. In some embodiments, capillary forces on each of the species within the fluid (e.g., analytes, antigens) can be balanced by electrical forces applies by the electrodes. Species of different electrical-charge-to-mass ratios may travel different distances within the capillary under the influence of electrical field applied by the electrodes, resulting in separation of different species within the fluids.

The techniques described herein also provide for a micro/nano-aliquoting fluidic system. This fluidic system can provide a mechanism for aliquoting, separating, sorting, and/or distributing biological specimens for biological assays. These biological assays may be useful for detecting, for example, biomarkers in human blood. The fluidic system can reside on a substrate (e.g., a carrier chip manufactured using micro/nano-technology) for small-scale aliquoting, separating, sorting, and/or distributing biological specimens. In some embodiments, the fluidic system can handle biological specimen-carrying volumes of a fraction of micro-liter (e.g., a nanoliter), or smaller.

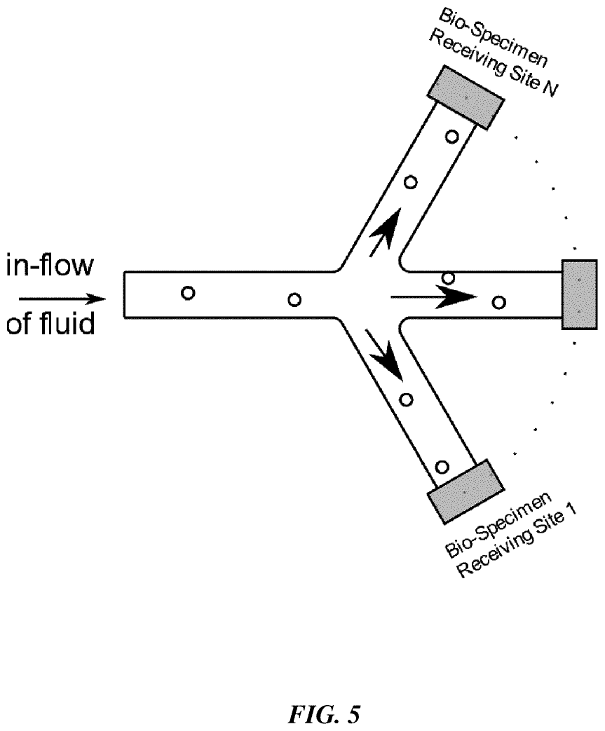

In some embodiments, the micro/nano-aliquoting system can separate analytes in an input tube (e.g., a capillary tube) fluidically connected to a junction into a plurality of output tubes (e.g., a first output tube, a second output tube, and/or a third output tube) to reach an assay at the end of each output tube, such a bio-specimen receiving site, as shown in FIG. 5. In some embodiments, some and/or all of the bio-specimen receiving sites are equally spaced from each other. That is to say, in some cases, the bio-specimen receiving sites are equidistant from each other. This may allow equal volumes of a sample from the inlet tube to be distributed among the outlet tubes such that arrival to the bio-specimen receiving sites is temporally aligned. However, in some embodiments, some and/or all of the bio-specimen regions are of disparate or different distances from one another. In this case, arrival of equal volumes of a sample are temporally spaced, which may allow for sequential arrival of volumes of the sample at various bio-specimen receiving sites fluidically connected to individual outlet tubes. In some embodiments, the dimensions of the tubes (e.g., an inlet tube, an outlet tube) may be of micro or nano-size and may be adjacent to a micro-sized substrate. The inventors have recognized and appreciated that such small-scale dimensions may allow for the collection of small volumes of sample (e.g. 100 nL, 10 nL, 1 nL of sample or less) while still providing accurate separation and analyte determination.

As described above, the micro- and/or nano-sized dimensions of the aliquoting fluidic systems described herein may advantageously allow for the separating of micro- or nano-volumes of a liquid (e.g. a fluid, a sample). Accordingly, in some embodiments, a cross-section diameter of a tube (e.g., a capillary tube, an inlet tube, an outlet tube, and the like) is no greater than approximately 1000 microns, no greater than 750 microns, no greater than 500 microns, no greater than 250 microns, no greater than 100 microns, no greater than 80 microns, no greater than 60 microns, no greater than 50 microns, no greater than 40 microns, no greater than 25 microns, no greater than 10 microns, no greater than 1 micron, no greater than 0.5 microns, or no greater than 0.1 microns. In some embodiments, a cross-section diameter is approximately at least 0.1 microns, at least 0.5 microns, at least 1 micron, at least 10 microns, at least 25 microns, at least 40 microns, at least 50 microns, at least 60 microns, at least 80 microns, at least 100 microns, at least 250 microns, at least 500 microns, at least 750 microns, or at least 1000 microns. Combinations of the above-reference ranges are also possible (e.g., no greater than approximately 250 microns and at least 50 microns). Other ranges are possible.

Nano-scale tube dimensions are also possible. Accordingly, in some embodiments, a cross-section diameter of a tube (e.g., a capillary tube, an inlet tube, an outlet tube, and the like) is no greater than approximately 100 nanometers, no greater than 90 nanometers, no greater than 80 nanometers, no greater than 70 nanometers, no greater than 60 nanometers, no greater than 50 nanometers, no greater than 40 nanometers, no greater than 30 nanometers, no greater than 20 nanometers, no greater than 10 nanometers, or no greater than 5 nanometers. Smaller nano-sized dimensions may also be possible, for example no greater than 1 nanometer (or no greater than 0.9 nanometers, no greater than 0.8 nanometers, no greater than 0.7 nanometers, no greater than 0.6 nanometers, no greater than 0.5 nanometers, or smaller). In some embodiments, a cross-section diameter is approximately at least 5 nanometers, at least 10 nanometers, at least 20 nanometers, at least 30 nanometers, at least 40 nanometers, at least 50 nanometers, at least 60 nanometers, at least 70 nanometers, at least 80 nanometers, at least 90 nanometers, or at least 100 nanometers. Combinations of the above-reference ranges are also possible (e.g., no greater than approximately 100 nanometers and at least 10 nanometers). Other ranges are possible.

In some embodiments, several inlet tubes connected to a plurality of analyte sources (e.g., bio-specimen sources) may join to a fluidically connected junction where analytes may be combined and directed towards one or more bio-specimen receiving sites.

In some embodiments, an inlet tube may carry a fluid (e.g., a sample) carrying bio-specimens towards a fluidically connected junction. The fluid can flow at a non-trivial (e.g., non-zero) flowrate through a manifold comprising at least one inlet tube fluidically connected to a junction where the manifold carries multiple outlet tubes leading to up to a plurality of receiving sites (e.g., bio-specimen receiving sites). Each receiving site can carry at least one bio-assay and can be of equal distance to the junction between inlet and outlets tubes. In certain embodiments, inlet/outlet tubes have equal diameter. In some embodiments, arrival of equal volume of bio-specimens of equal attributes at each receiving site is temporally aligned.

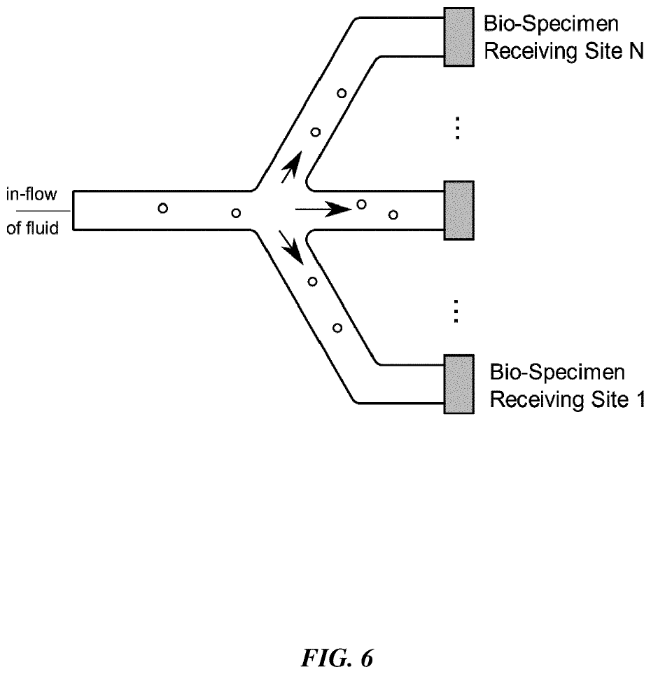

In some embodiments, an inlet tube may carry a fluid (e.g., a sample) carrying bio-specimens towards a fluidically connected junction. The fluid can flow at a non-trivial (e.g., non-zero) flowrate through a manifold comprising at least one inlet tube fluidically connected to a junction where the manifold carries multiple outlet tubes leading to up to a plurality of receiving sites (e.g., bio-specimen receiving sites). Each receiving site may carry at least one bio-assay or a bio-positive/negative control. In some embodiments, each receiving site can be engineered to have disparate distances to the junction between inlet and outlets whereby arrival of equal volumes of bio-specimens of equal attributes at each receiving site can be temporally spaced as a result of the engineered distance between the receiving sites and the junction. An example of such an embodiment is schematically shown in FIG. 6.

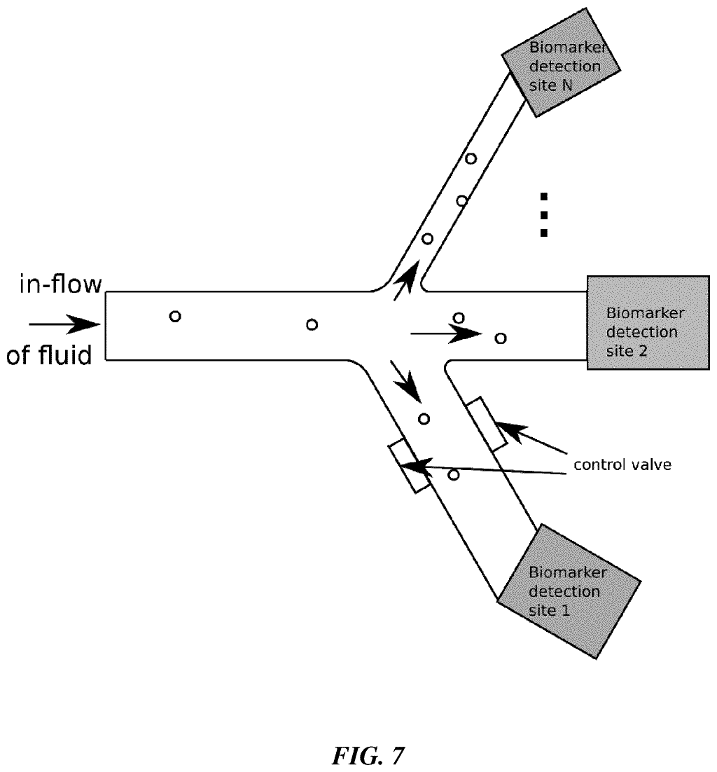

FIG. 7 provides a schematic exemplary system diagram of a micro-aliquoting system using techniques described herein. A fluid sample, such as serum or filtered blood may flow through the inlet tube with or without biomarkers. The micro/nano-fluid channel(s) can have manifold outlets leading to each individual biomarker detection site. The outlet tubes may have equal or disparate diameters, and biomarkers approaching each detection site may approach with equal or disparate rates. In some embodiments, the flow rate in an outlet tube can be adjusted if needed, such by use of a control valve. Each assay at each detection site can differ from each other (e.g., a bio-assay, a positive control, a negative control, and/or the like).

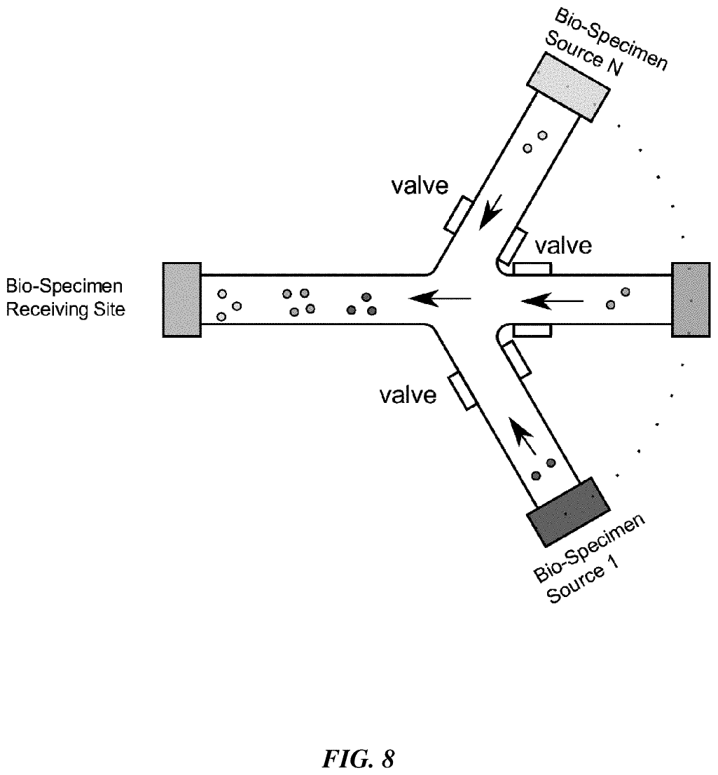

In some embodiments, the techniques provide for a micro-distribution system. The system may comprise a plurality of bio-specimen sources which may receive a fluid sample (e.g., serum, filtered blood, and/or the like). The micro-fluid manifold may have a plurality of inlets connecting each individual bio-specimen source. In addition, the micro-fluid manifold has at least one outlet accessible by each inlet at the inlet/outlet junction. Access from each inlet to the junction can be controlled by a control valve (e.g., a micro-valve), as schematically depicted in FIG. 8. A non-trivial pressure may be applied from each source site and checked by the associated valve. In some embodiments, bio-specimens from the sources are sequentially interweaved into the outlet via collaborative operations of individual valves.

The following examples are intended to illustrate certain embodiments of the present invention, but do not exemplify the full scope of the invention.

### Example 1

The following example describes the quantitation of antigen concentrations using standard curves constructed using known concentrations of a certain antigen. The antigen binding entity is an anti-CRP antibody.

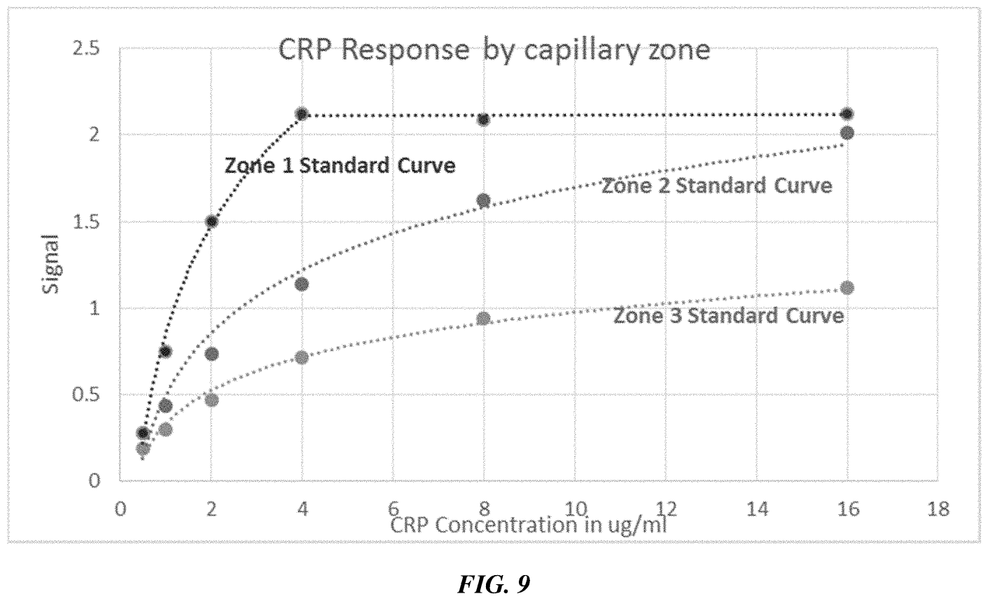

The capillary tube was constructed of six conjoined sections, with sections 1, 3, and 5 (zones 1, 3 and 5) containing bound anti-CRP and sections 2, 4 and 6 without bound anti-CRP. Using the detection method chosen, standard curves at CRP concentrations of physiological interest (0.5 mg/L to 10 mg/L) in blood serum, or plasma (the biological fluids to be used in the test)) were constructed using spiked samples. The zone or zones with the largest dynamic range (largest difference in signal between the lowest concentration sample in the physiological range and the highest concentration sample in the physiological range and the ability to accurately discriminate between all concentrations in the range) were chosen for CRP quantitation, as shown in FIG. 9. In some cases, two zones may be used for quantitation, one for low CRP concentrations and one for high CRP concentrations.

As shown in the plots in FIG. 9, the signal from zone 1 gives a very sensitive CRP assay, but with a very narrow dynamic range of approximately 0.25 to 4 mg/L after. The signal from zone 2 gives an assay of lower sensitivity, but still in the desired measurement range with a greatly improved dynamic range from approximately 0.5 mg/L to 16 mg/L. Finally, the signal from zone 3 has a poor dynamic range because it is unable to accurately able to discriminate between 8 and 16 mg/L.

It was also be possible to use two zones for CRP quantitation. Signal from zone 1 could be used to quantitate CRP from 0 to approximately 4 mg/L and zone 2 could be used to quantitate CRP between approximately 4 and 16 mg/L

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A capillary tube, comprising:
a first section located within the capillary tube, the first section comprising a first material;
a plurality of species-binding entities attached to the first material; and
a sensor adjacent to at least some of the plurality of species-binding entities.

2. The capillary tube of claim 1, comprising a second section located within the capillary tube, the second section comprising a second material.

3. The capillary tube of claim 1, wherein the second material is configured to prevent attachment of the plurality of species-binding entities.

4. The capillary tube of claim 1, wherein the plurality of species-binding entities comprises antibodies.

5. The capillary tube of claim 1, wherein the sensor is arranged and configured to detect a change in an electric field proximate the plurality of species-binding entities.

6. The capillary tube of claim 1, further comprising a sample, wherein the sample comprises at least one species configured to bind to the plurality of species-binding entities.

7. A capillary tube, comprising:
a first section located within the capillary tube, the first section comprising a first material;
a first plurality of species-binding entities attached to the first material,
wherein the plurality of species-binding entities are configured to selectively release from the first material; and
a sensor proximate to a distal end of the capillary tube.

8. The capillary tube of claim 1, wherein the first material is configured to release at least some of the first plurality of species-binding entities.

9. The capillary tube of claim 1, wherein binding of a species to the first plurality of species-binding entities results in release of at least some of the first plurality of species-binding entities from the first section.

10. The capillary tube of claim 1, comprising a second section located within the capillary tube, the second section comprising a second material, wherein the second material is configured to prevent attachment of the first plurality of species-binding entities.

11. The capillary tube of claim 1, further comprising a third section within the capillary tube, the third section comprising a third material.

12. The capillary tube of claim 1, comprising a second plurality of species-binding entities.

13. A capillary tube, comprising:
a first section located within the capillary tube, the first section comprising:
a first material; and
a first plurality of species-binding entities attached to the first material, wherein the first plurality of species-binding entities is configured to selectively release from the first material;
a second section located within the capillary tube, the second section comprising a second material, wherein the second material is different than the first material and configured to prevent attachment of the first plurality of species-binding entities;
a third section comprising a second plurality of species-binding entities configured to selectively bind a species, the species bound to the first plurality of species-binding entities; and
a sensor adjacent to at least some of the second plurality of species-binding entities.

14. The capillary tube of the preceding claim, further comprising a third plurality of species-binding entities.

15. The capillary tube of claim 1, wherein
the first material is configured to release at least some of the first plurality of species-binding entities.

16. The capillary tube of claim 1, wherein binding of a species to the first plurality of species-binding entities results in release of at least some of the first plurality of species-binding entities from the first material.

17-18. (canceled)

19. A system, comprising:
a capillary tube comprising a proximal end and a distal end,
wherein the capillary tube comprises a plurality of species-binding entities attached to at least a first section of the capillary tube;
a first electrode proximate the proximal end;
a second electrode; and
a power supply connected to the first electrode and the second electrode.

20-24. (canceled)

25. A system, comprising:
an inlet tube;
a junction fluidically connected to the inlet tube; and
a first outlet tube, a second outlet tube, and a third outlet tube each fluidically connected to the junction,
wherein the inlet tube, the first outlet tube, the second outlet tube, and the third outlet tube are no greater than approximately 1 micron in a cross-section diameter.

26-30. (canceled)

31. A system, comprising:
an inlet tube;
a junction fluidically connected to the inlet tube; and
a first outlet tube, a second outlet tube, and a third outlet tube each fluidically connected to the junction,
wherein the inlet tube, the first outlet tube, the second outlet tube, and the third outlet tube are no greater than approximately 100 nanometers in a cross-section diameter.

32-37. (canceled)

38. A system, comprising:
a first source comprising a first inlet;
a second source comprising a second inlet;
a third source comprising a third inlet;
a junction fluidically connecting the first inlet, the second inlet, and the third inlet;
an outlet tube fluidically connected to the junction; and
a receiving site proximate to a terminal end of the outlet tube,
wherein the first inlet, the second inlet, the third inlet, and/or the outlet tube have a cross-sectional diameter of no greater than approximately 1 micron.

39-42. (canceled)
